(19) United States
(12) Patent Application Publication
(10) Pub. No.: US 2009/0126145 A1
(43) Pub. Date: May 21, 2009

(54) Vacuum cleaner with sterilizing lamp

(76) Inventors: Daniel D'Agostino, Austin, TX (US); Robert Plekker, Bellingham, WA (US); Kyle Windemuller, Hudsonville, MI (US)

Correspondence Address: KRIEGSMAN & KRIEGSMAN, 30 TURNPIKE ROAD, SUITE 9, SOUTHBOROUGH, MA, 01772, US

(21) Appl. No.: 11/985,562

(22) Filed: Nov. 15, 2007

(51) Int. Cl. A47L 7/00

(52) U.S. Cl. 15/339; 15/246.3

(57) ABSTRACT

A vacuum cleaner for simultaneously cleaning and sterilizing a surface includes a vacuum cleaner housing, a plurality of wheels for supporting the vacuum cleaner housing on the surface, a main power switch for electrically connecting the vacuum cleaner to an external source of power, a motor coupled to the main power switch and a rotary brush coupled to the motor. The motor includes a suction fan. A UV lamp housing assembly is mounted inside the vacuum cleaner housing and urged downward toward the surface. The UV lamp assembly includes a UV lamp for sterilizing the surface at the same time as it is being cleaned. A UV light gasket assembly is fixedly mounted underneath the UV lamp assembly and includes a track of conductive material having a plurality of gaps and a plurality of interlock switches for closing the gaps, one interlock switch being associated with each gap. Each interlock switch includes a cap and an interlock contact of conductive material which is fixed to the cap. Each cap is movable between a depressed position where its interlock contact bridges the gap associated with the switch and a non-depressed position where its interlock contact is spaced from its associated gap. A compression spring on each cap urges the cap downward to its non-depressed position. When all of the gaps in the track are bridged by the interlock switches, the UV lamp is electrically connected to the main power source and is energized when the main power switch is turned on. The gaps are positioned on the track and the track is disposed within the vacuum cleaner housing such that tilting the vacuum cleaner in any direction relative to the surface or lifting the vacuum cleaner off of the surface will cause at least one of the caps to be moved to its non-depressed position along with its associated interlock contact, thereby disconnecting the UV lamp from the main power source.

### BACKGROUND OF THE INVENTION

The present invention relates generally to vacuum cleaners and more particularly to a vacuum cleaner which includes an ultraviolet (UV) lamp for sterilizing a surface such as a rug at the same time as the surface is being cleaned.

Vacuum cleaners which include an ultraviolet lamp for sterilizing a surface at the same time as the surface is being cleaned are well known in the art. One problem with this type of vacuum cleaner is that the ultraviolet light emitted by the lamp can cause injury to the person using the cleaner.

In U.S. Pat. No. 2,590,152 to J. P. Buckey there is disclosed a vacuum cleaner which includes an ultraviolet lamp in the nozzle of the cleaner for directing ultraviolet rays against the surface being cleaned in order to sterilize the surface at the same time as it is being cleaned. In one embodiment of the invention described in the patent, the ultraviolet lamp is electrically connected to the main switch of the cleaner through a mercury switch which is normally closed but which opens when the cleaner is tilted in any direction. A problem with this arrangement is that the mercury switch will not open if the cleaner is lifted off a surface but is not tilted. In another embodiment of the invention described in the patent, the ultraviolet lamp is electrically connected to the main switch of the cleaner by a micro-switch which is under control of a spring arranged such that the micro-switch is opened when the cleaner is lifted from the floor regardless of whether or not it is tilted.

In U.S. Pat. No. 2,681,467 to R. Guyer there is disclosed a vacuum cleaner having a brush rotated by the vacuum cleaner motor and an ultraviolet lamp located above the brush. The lamp is connected to the power source used to power the motor. The problem with this arrangement is that as long as the vacuum cleaner motor is on the lamp will be energized. Tilting or lifting the vacuum cleaner off the surface will not cause the lamp to be de-energized.

In U.S. Pat. No. 5,233,723 there is disclosed a vacuum cleaner which includes an ultraviolet lamp which is arranged to direct ultraviolet rays onto the surface being cleaned and also into a suction nozzle in the cleaner. No mention is made as to how or when the lamp is energized or de-energized.

In U.S. Pat. No. 4,907,316 there is disclosed a vacuum cleaner which includes an ultraviolet radiator installed in a suction nozzle and a movement sensor coupled to the ultraviolet radiator for switching off the radiator when the nozzle is not moved for a predetermined period of time.

Other patents and patent applications disclosing vacuum cleaners which include an ultraviolet lamp are U.S. Pat. No. 3,975,790 to L. Patterson; U.S. Pat. No. 6,239,492 to K. Limura; U.S. Patent Publication No. US2005/0091785 to S. Yuen; U.S. Patent Publication No. U.S. 2003/0131439 to S. Wen; Canadian Patent Application 2,094,678 to U. F. Hung; U.K. Patent Application GB 2,412,059 to J. Yuen; PCT International Application No. PCT/YU2005/000019 to D. Peruricic and Patent Abstracts of Japan Publication No. 2005-013526 to S. Hideyuki.

It is an object of this invention to provide a vacuum cleaner having an ultraviolet lamp for sterilizing a surface as it is being cleaned and which overcomes the disadvantages of the prior art vacuum cleaners of this type with regard to when and how the ultraviolet lamp is energized and de-energized.

### SUMMARY OF THE INVENTION

According to this invention, there is provided a vacuum cleaner which includes a UV lamp for sterilizing a surface as it is being cleaned, wherein the UV lamp is automatically energized when the main power switch is turned on and the cleaner is disposed, either stationary or moving, on the surface and wherein the UV lamp is automatically de-energized when either the main power switch is turned off or the vacuum cleaner is tilted in any direction relative to the surface or is lifted off the surface.

The vacuum cleaner includes a main housing, a plurality of wheels on the main housing for supporting the vacuum cleaner on the surface to be cleaned, a main power switch for electrically connecting the vacuum cleaner to an external source of power, a motor inside the main housing and electrically connected to the main power switch, a brush housing, a rotary brush mounted in the brush housing and coupled to the motor for agitating dirt on the surface, the motor including a suction fan for creating suction to suck in dirt on the surface agitated by the rotary brush, a UV lamp assembly, a UV lamp within the UV lamp assembly, a UV light gasket assembly below the UV lamp assembly, the light gasket assembly including a grate, the grate having an opening through which light from the UV lamp can pass through and strike the surface to sterilize the same, an electrical circuit on the grate for electrically connecting the main power switch to the UV lamp, the electrical circuit comprising a generally oval shaped track of conductive material having a plurality of gaps and a plurality of interlock switches for opening and closing the gaps, one interlock switch being associated with below each gap, each interlock switch having a cap, a contact fixed to the cap and a spring, the cap being movable up and down between a depressed position wherein the contact bridges the gap and a non-depressed position wherein the contact is spaced from the gap, the spring in each interlock switch urging the contact away from the track, wherein, when the cap in each one of the interlock switches is in its depressed position thereby closing all the gaps in the track, current will flow from the main power switch to the UV lamp and, wherein the gaps are located on the track and the track is located relative to the housing such that tilting the vacuum cleaner relative to the surface in any direction or lifting the vacuum cleaner off the surface will cause at least one of the caps to be moved to its non-depressed position thereby disconnecting the UV lamp from the main power switch.

Various other features and advantages will appear from the description to follow. In the description, reference is made to the accompanying drawings which form a part thereof, and in which is shown by way of illustration, at least one embodiment for practicing the invention. This embodiment will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings wherein like references numerals represent like parts:

FIG. 1 is a perspective view taken from the side of a vacuum cleaner constructed according to this invention;

FIG. 2 is a perspective view of the vacuum cleaner as shown in FIG. 1 but with the main housing broken away at the right;

FIG. 3 is a perspective view of the vacuum cleaner as shown in FIG. 1 but with the main housing broken away at the left;

FIG. 4 is a right side view of the vacuum cleaner shown in FIG. 1 with the main housing broken away;

FIG. 5 is an enlarged fragmentary perspective view of the vacuum cleaner as shown in FIG. 1;

FIG. 6 is a perspective view taken from the bottom of the vacuum cleaner shown in FIG. 1;

FIG. 7 is a perspective view shown upside down of the light housing assembly in the vacuum cleaner shown in FIG. 1;

FIG. 8 is an exploded perspective view shown upside down of the light housing assembly in the vacuum cleaner shown in FIG. 7;

FIG. 9 is a perspective view shown upside down of the light gasket assembly shown in the vacuum cleaner in FIG. 1;

FIG. 10 is a fragmentary perspective view taken from the bottom of the light gasket assembly in FIG. 9 with one of the interlock switches removed;

FIG. 11 is an exploded perspective view shown upside down of the light gasket assembly shown in FIG. 9;

FIG. 12 is a perspective view of one of the interlock switches shown in FIG. 9;

FIG. 13 is an exploded perspective view of the interlock switch shown in FIG. 12;

FIG. 14 is a side view partly broken away in section of the light housing assembly and light gasket assembly shown in the vacuum cleaner in FIG. 1 with the interlock switches in a non-depressed position;

FIG. 15 is a side view partly broken away in section of the light housing assembly and light gasket assembly shown in the vacuum cleaner in FIG. 1 with the interlock switches in a depressed position;

FIG. 16 is an enlarged fragmentary perspective view of one of the interlock switches in its non-depressed position;

FIG. 17 is an enlarged fragmentary perspective view of one of the interlock switches in its depressed position, and

FIG. 18 is an electrical schematic showing how the motor and UV lamp in the vacuum cleaner in FIG. 1 are connected to the main power switch.

### DETAILED DESCRIPTION OF THE PREFERRED EMBODIMENT

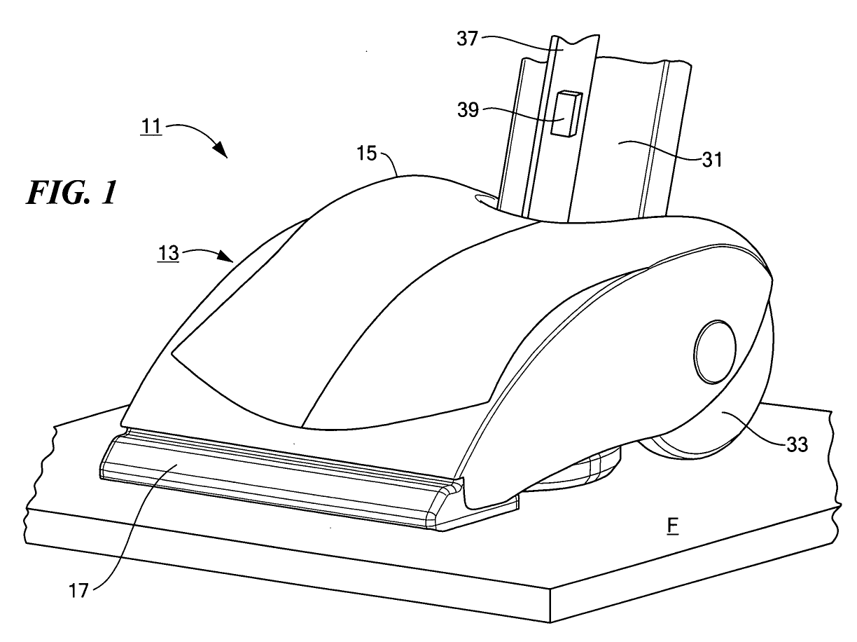

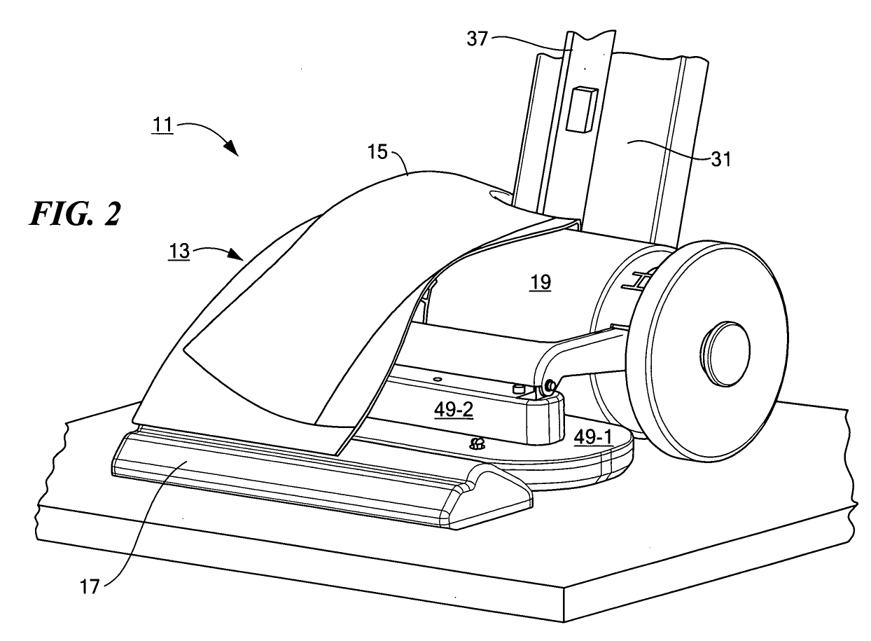

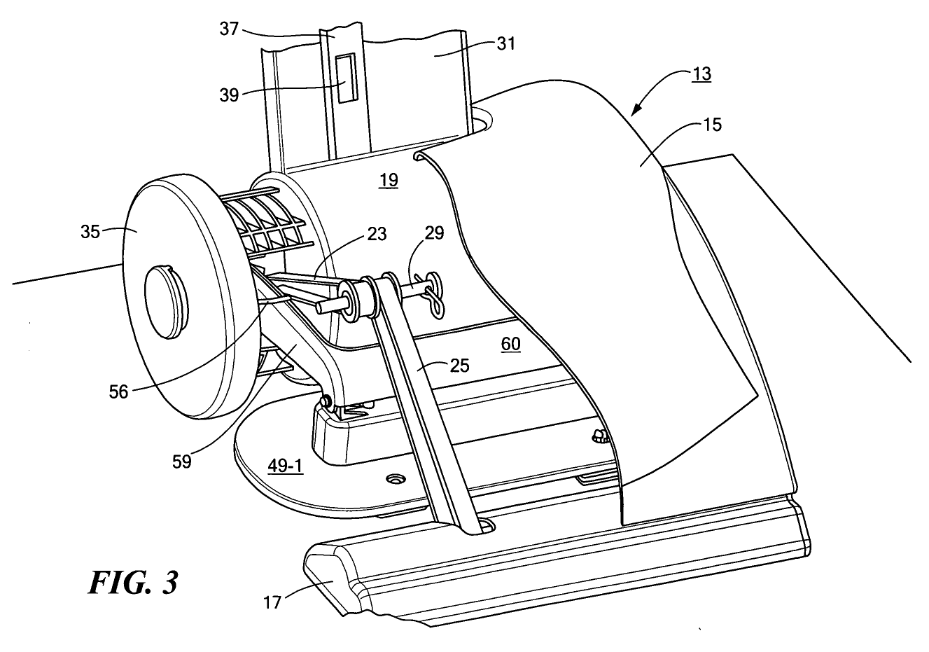

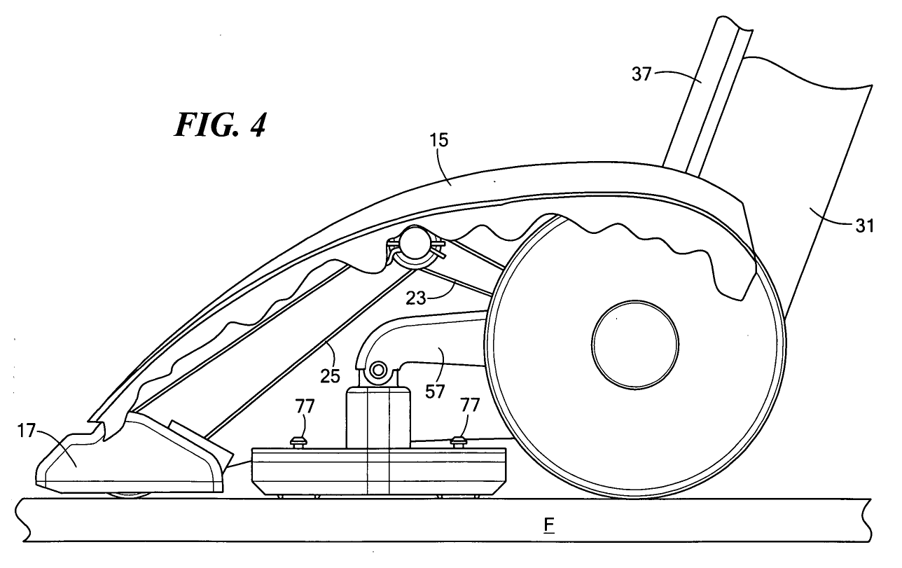

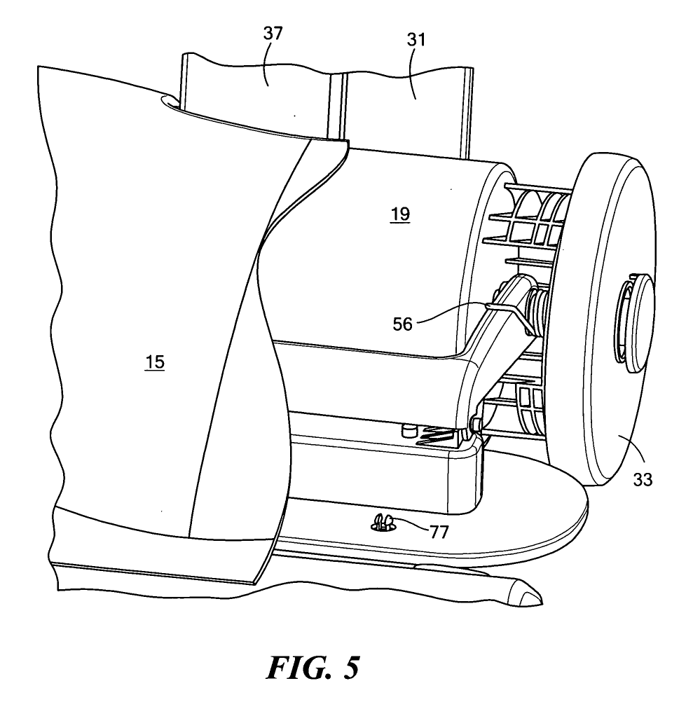

Referring now to the drawings and first to FIG. 1 through FIG. 6, there is shown a vacuum cleaner constructed according to this invention and identified by reference numeral 11. Vacuum cleaner 11 is shown seated on a floor F; however, it should be noted that vacuum cleaner 11 can be used to clean other surfaces and is not limited to cleaning floors.

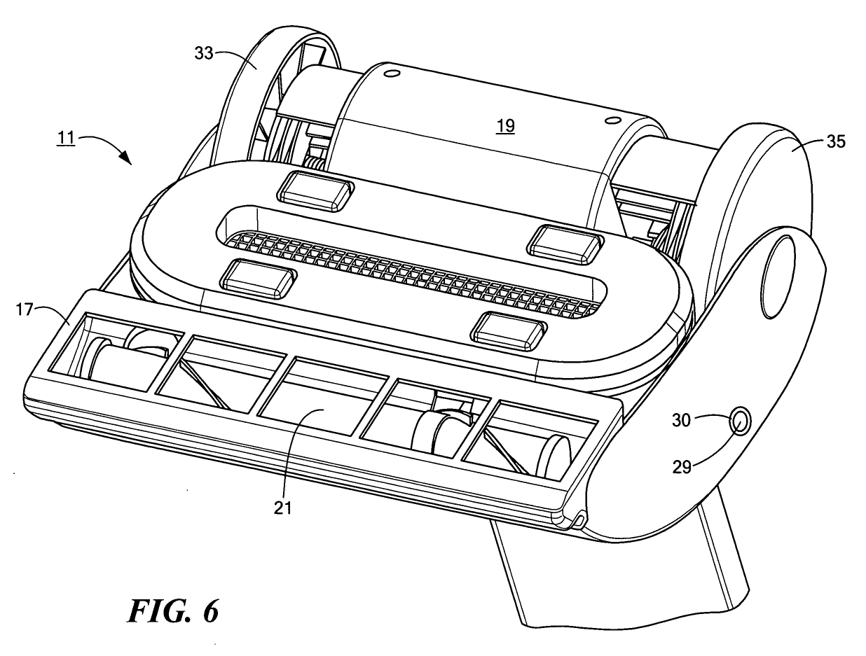

Vacuum cleaner 11 comprises a vacuum cleaner housing 13 which is made up of two parts, namely, a main housing 15 and a brush housing 17. Brush housing 17 is shaped to define a suction nozzle, is disposed in front of main housing 15 and is attached to main housing 17 by any suitable means (not shown). An electric motor 19 having a suction fan on its shaft for creating suction is rotably mounted inside main housing 15 and a rotary brush 21 is mounted inside brush housing 17. A pair of belts 23 and 25 drive the rotary brush 21. Belt 23 is mounted on axle 27 of motor 18 at one end and a spindle 29 at the other end. Spindle 29 is rotably mounted by a bearing 30 in Housing 15 (See FIG. 6). Belt 25 is mounted on spindle 29 at one end and rotary brush 21 at the other end.

Housing 15 is shaped to provide air passages (not shown) which extend from brush housing 17 rearwardly past motor 19 and communicate with a filter bag 31.

A pair of wheels 33 and 35 are rotably mounted on housing 15 for supporting vacuum cleaner 11 on floor F or other surface to be cleaned in the usual manner. A handle 37 is coupled to housing 13 to enable vacuum cleaner 11 to be easily moved and a main power switch 39 for electrically connecting vacuum cleaner 11 to an external source of power is mounted on handle 37. For simplicity, the wires connecting main power switch 39 to motor 19 are not shown.

In the operation of vacuum cleaner 11, rotary brush 21 agitates dirt on the floor F being cleaned while the suction fan on motor 19 sucks up dirt agitated by rotary brushy 21 and then exhausts the dirt sucked up into filter bag 31. The weight of main housing 15 of vacuum cleaner 11 keeps rotary brush 21 on floor F.

According to the present invention, a sterilizing mechanism 41 is provided for sterilizing the surface at the same time as it is being cleaned. Sterilizing mechanism 41 includes a UV light assembly 43 and a UV light gasket assembly 45.

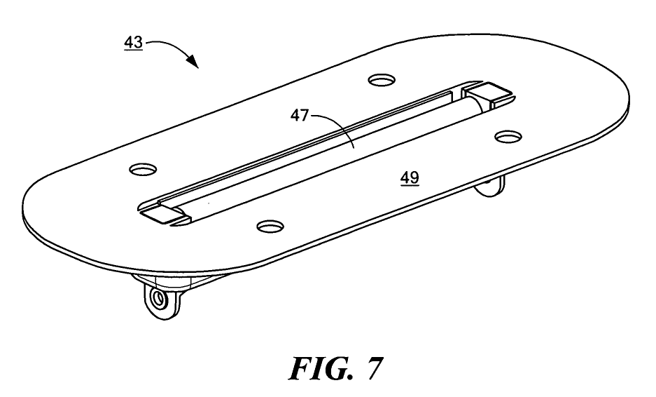

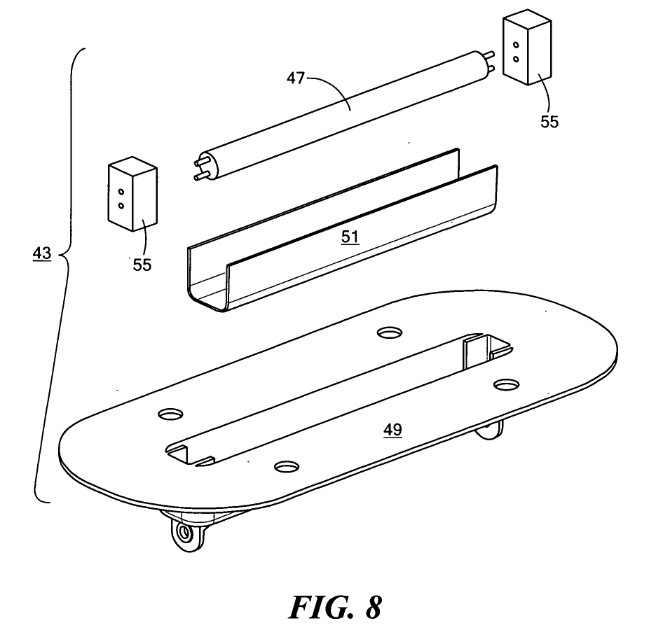

UV light assembly 43, see FIGS. 7 and 8, includes a UV lamp 47 for emitting UV radiation, a lamp housing 49 for holding UV lamp 47, a reflector 51 for reflecting downward UV light emitted from UV lamp 47 and a pair of foam spacers 53 and 55 for holding lamp 47 in place on lamp housing 49. Lamp housing 49 is pivotally attached to the axle of wheels 33 and 35 by a pair of arms 57 and 59 which are connected to each other by a cross bar 60. Lamp housing 49 includes a base portion 49-1 and a lamp holding portion 40-2.

Torsion springs 56 mounted on either side of motor 19 continuously push arms 57 and 59 in a downward direction, causing UV light assembly 43 and along with it UV light gasket assembly 47 to be biased downward.

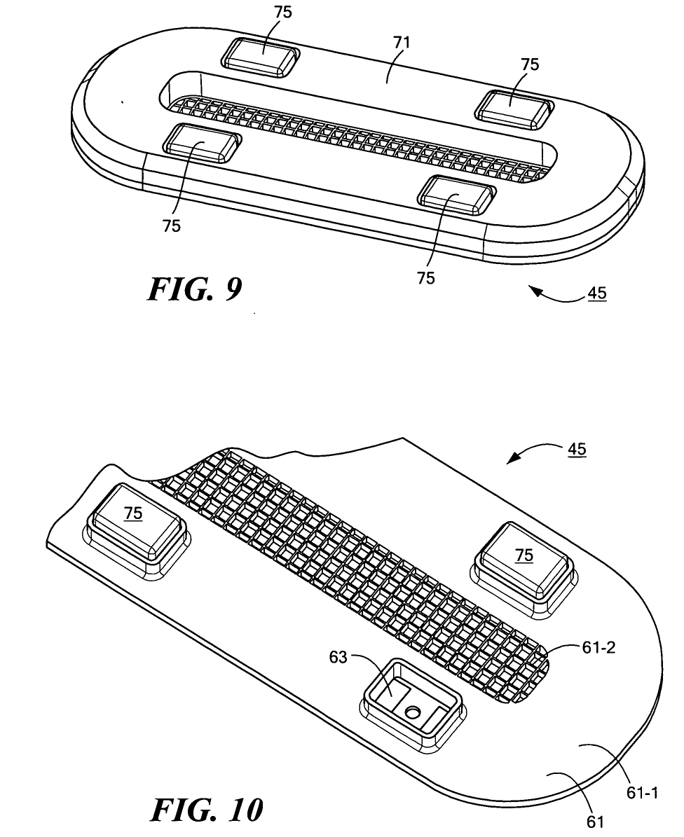

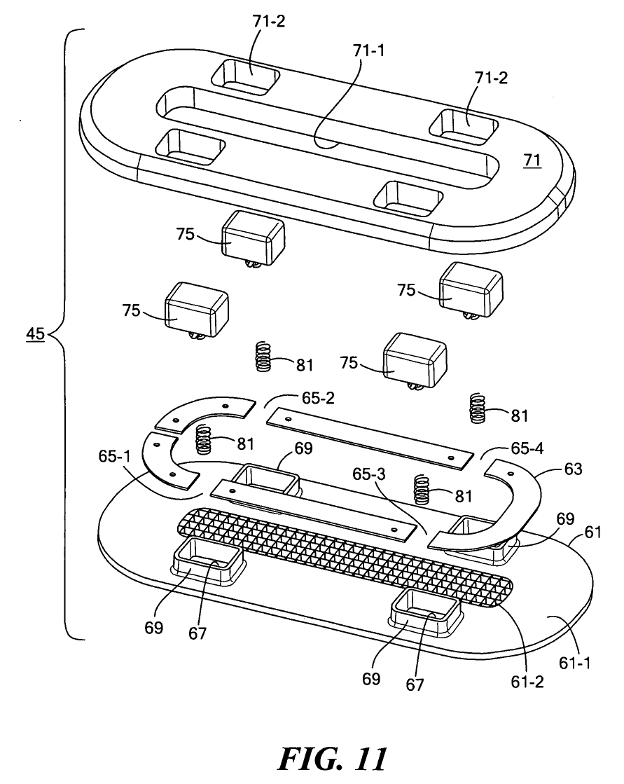

UV light gasket assembly 45, see FIGS. 9 through 11, includes an oval shaped grate 61 made of non-conductive material such as plastic. Grate 61 is secured by any suitable means, not shown, to the bottom of lamp housing 49. The outer portion 61-1 of grate 61 is solid while the inner portion 61-2 of grate 61 is shaped to define an array of openings to allow light to pass through. Light gasket assembly 45 also includes an oval shaped track 63 of conductive material is molded into grate 61. Track 63 has four gaps 65-1 through 65-4, gap 65-1 being at the front left portion of track 63, gap 65-2 being at the back left portion of track 63, gap 65-3 being at the front right portion of track 63 and gap 65-4 being at the back right portion of track 63.

The outer portion 61-1 of grate 61 has four rectangular openings 67, each opening 67 being aligned with one of the gaps 65 and being sized larger than its associated gap so that a portion of track 63 on either side of the gap is shown, see FIG. 10. An extruded rectangular wall 69 extends down from each opening 67. UV light gasket assembly 45 also includes an oval shaped foam gasket 71 is fixedly secured by any means, not shown, to the bottom surface of grate 61. Gasket 71 includes an opening 71-1 aligned with inner portion 61-2 of grate 61 and four openings 71-2 aligned with openings 67 in grate 61.

Finally, UV light gasket assembly 45 includes four interlock switches 73, one interlock switch 73 being movably disposed within each rectangular opening 67.

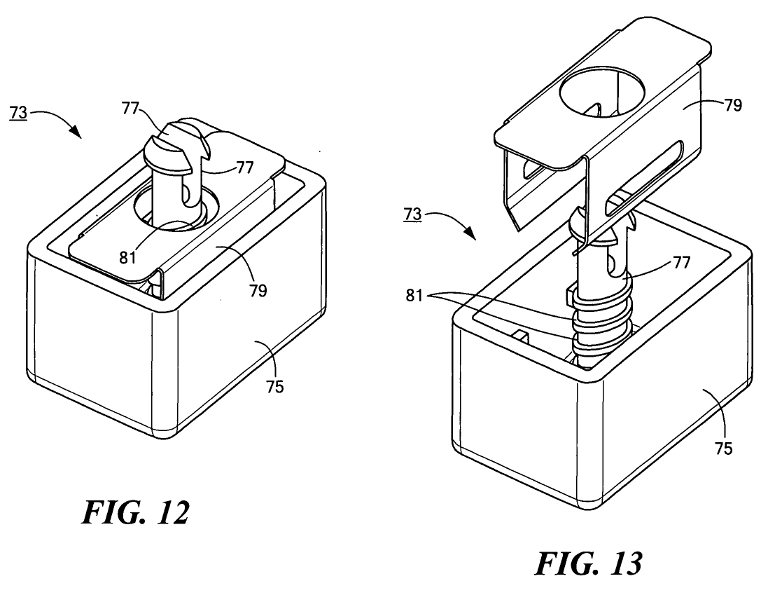

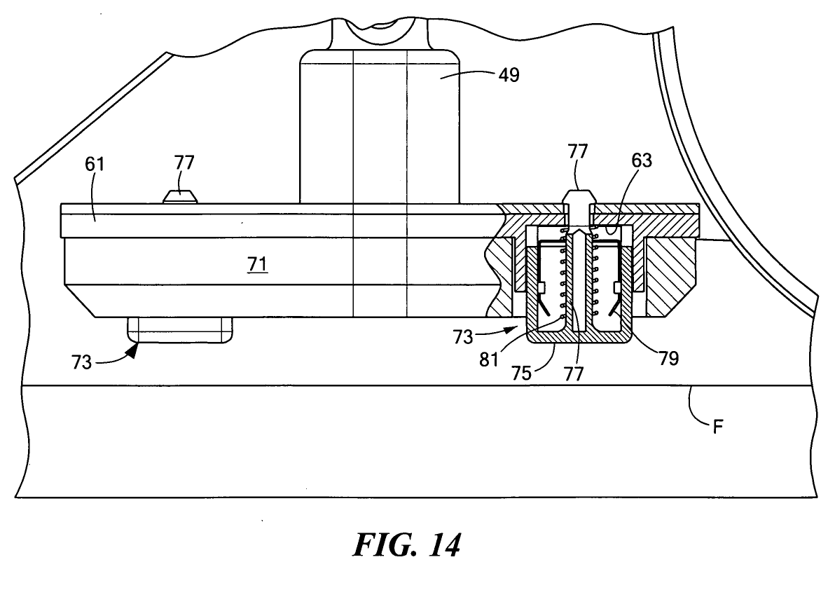

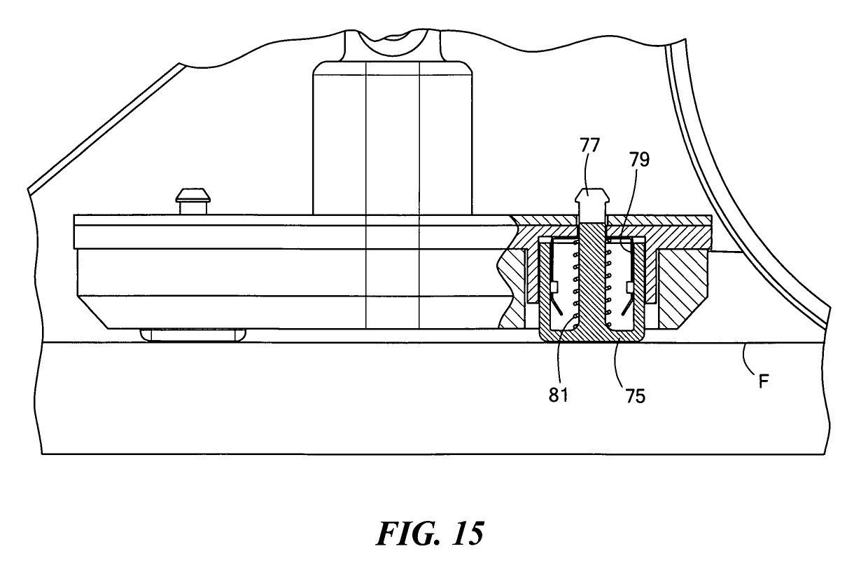

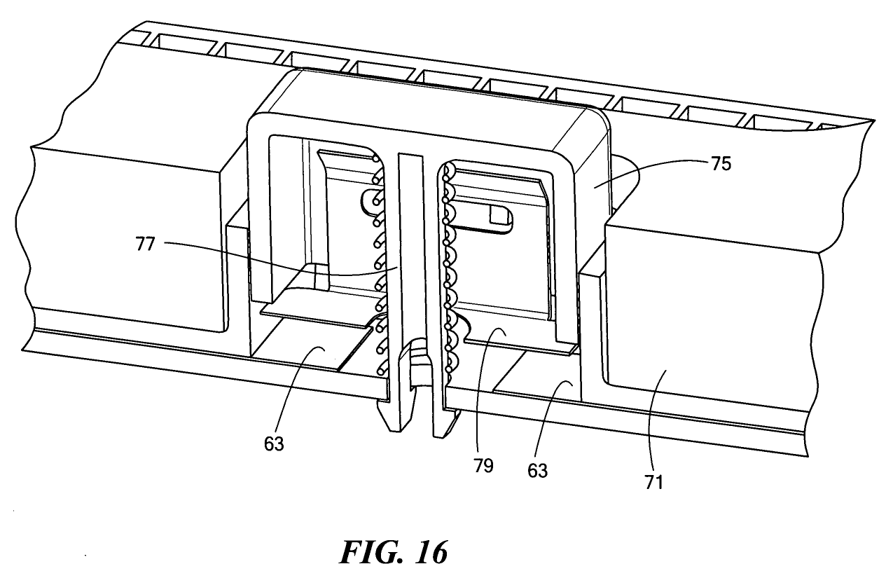

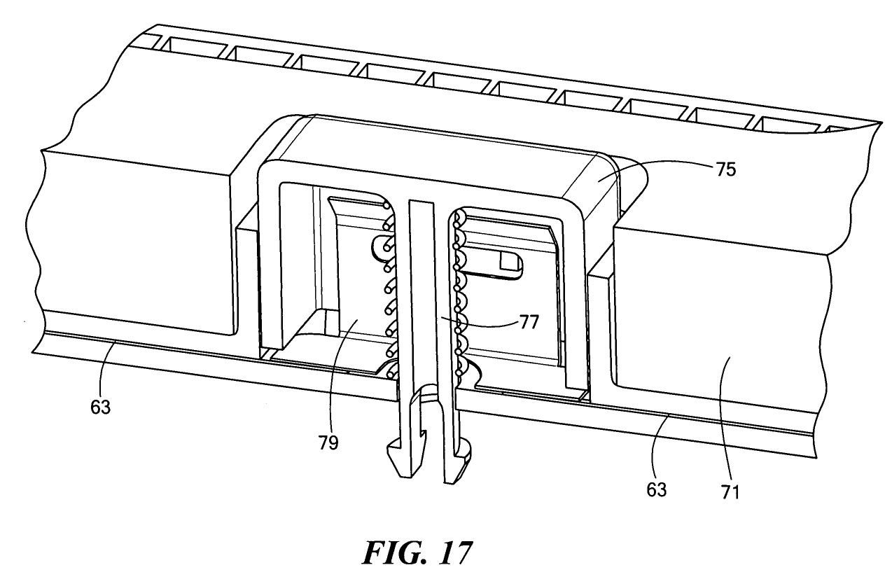

Interlock switch 73, which is shown in FIGS. 12 and 13, includes a cap housing 75 of non-conductive material, cap housing 75 having a projection pawl 77, a contact 79 of conductive material fixedly mounted in and a compression spring 81. Interlock switch 73 is movable up and down between a depressed position in which contact 79 bridges its associated gap, see FIG. 17 and a non-depressed position see FIG. 16 in which contact 79 does not bridge its associated gap. Compression spring 81 maintains cap 79 in a normally non-depressed position. When all four gaps 65 are bridged by the interlock switches 65, the circuit connecting UV lamp 47 to main power switch 39 will be closed and lamp 47 will be energized. On the other hand, when any one or more of the gaps are not bridged, the circuit will be open and lamp 47 will not be energized.

Sterilizing mechanism 41 operates in the following manner to energize and/or de-energize UV lamp 47.

When vacuum cleaner 11 is not disposed on a surface such as a floor, all of the interlock switches 73 will be in their non-depressed position. As a result, the circuit from the main power switch 39 to UV lamp 47 will be open and UV lamp will not be energized regardless of whether main power switch 39 is in the “on” position of the “off” position.

On the other hand, when main power switch 39 is in the “on” position and vacuum cleaner is disposed on a surface, and either station any or moving, arms 57 and 59 will be biased downward such that all four interlock switches 73 will be pushed into their depressed position, closing all the gaps 65 and thereby energizing UV lamp 47. If vacuum cleaner 11 is either lifted off the surface or tilted in any direction, at least one of the interlock switches 73 will not bridge its associated gap, thereby opening the circuit and de-energizing UV lamp 47.

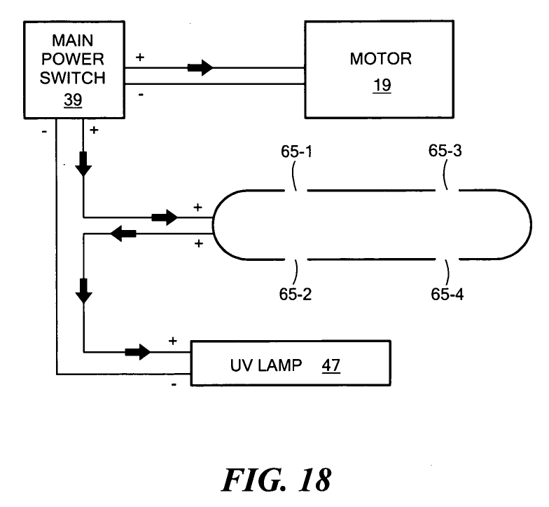

A schematic of the electrical circuit is shown in FIG. 18.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A vacuum cleaner comprising:
(a) a vacuum cleaner housing,
(b) a plurality of wheels on the vacuum cleaner housing for supporting the vacuum cleaner on a surface being cleaned,
(c) a main power switch for electrically connecting the electric vacuum cleaner to an external source of power,
(d) a motor inside the vacuum cleaner housing and electrically connected to the main power switch,
(e) an impeller on the motor for creating suction inside the vacuum cleaner housing,
(f) a rotary brush coupled to the motor,
(g) a UV lamp for sterilizing the surface at the same time as it is being cleaned,
(h) an electrical circuit for electrically connecting the main power switch to the UV lamp, the electrical circuit comprising a track of conductive material having a plurality of gaps and a plurality of interlock switches for bridging the gaps, one interlock switch being associated with each gap,
(i) each interlock switch having a cap, a contact of conductive material fixed to the cap and a spring, the cap being movable between an up position wherein the cap is depressed and the contact bridges the gap and a down position wherein the cap is non-depressed and where the contact is spaced from the gap, the spring urging the cap down toward its non-depressed position,
(m) wherein, when the cap in each one of the interlock switches is in its depressed position all the gaps in the track will be closed thereby enabling current to flow in the track from the main power switch to the UV lamp and, wherein the gaps are disposed relative to the housing such that tilting the vacuum cleaner in any direction relative to the surface being cleaned or lifting the vacuum cleaner off the surface being cleaned will cause at least one of the caps to be moved to its non-depressed position along with its associated contact, thereby opening the electrical circuit and disconnecting the UV lamp from the main power source.

2. A vacuum cleaner for simultaneously cleaning and sterilizing a surface, the vacuum cleaner comprising:
(a) a housing,
(b) a plurality of wheels on the housing for supporting the vacuum cleaner on the surface being cleaned,
(c) a main power switch for electrically connecting the vacuum cleaner to an external source of power,
(d) a motor inside the housing and electrically connected to the main power switch, the motor having a suction fan on its shaft for creating suction inside the vacuum cleaner;
(e) a rotary brush mounted on the housing and coupled to the motor for agitating dirt on the surface,
(f) a UV lamp inside the housing for emitting UV radiation for sterilizing the surface at the same time as it is being cleaned, and
(g) an electrical circuit for electrically connecting the main power switch to the UV lamp, the electrical circuit being constructed such that it will open automatically if the vacuum cleaner is either tilted relative to the surface being cleaned or lifted off the surface.

3. The vacuum cleaner of claim 1, wherein the electrical circuit comprises:
(a) a track of conductive material having a plurality of gaps, and
(b) a plurality of interlock switches for bridging the gaps, one interlock switch being associated with each gap, the electrical circuit being closed when all the gaps are bridged,
(c) wherein, tilting the vacuum cleaner in any direction or lifting the vacuum cleaner off the surface will cause at least one of the interlock switches to be moved away from its gap thereby opening the electrical circuit.

4. The vacuum cleaner of claim 2 wherein each one of the electrical switches comprises:
(a) a cap,
(b) a contact of conductive material fixed to the cap, and
(c) a spring coupled to the cap,
(d) wherein, the cap is disposed above the track and is movable between an up position where the cap is non-depressed and the contact is spaced up from its associated gap and a down position where the cap is depressed and the contact bridges its associated gap, the spring urging the cap down towards its depressed position.

5. A vacuum cleaner for cleaning a surface and sterilizing the surface at the same time as it is being cleaned, the vacuum cleaner comprising:
(a) a housing,
(b) a motor inside the housing, the motor including a suction fan on its output shaft for creating suction inside the housing,
(c) a main power switch for electrically connecting the motor to an external source of power,
(d) the housing having a main section and a brush section,
(e) a brush assembly in the brush section and coupled to the motor,
(f) a UV lamp inside the main section of the housing for emitting UV radiation,
(g) an electrical circuit for electrically connecting the main power switch to the UV lamp, the electrical circuit comprising a track of conductive material having a plurality of gaps and a plurality of interlock switches for bridging the gaps, one interlock switch being associated with each gap, each interlock switch is being open when it is out of contact with the surface and being closed when it is in contact with the surface,
(h) wherein, the gaps on the track of conducting material are disposed about the housing such that tilting the vacuum cleaner in any direction relative to the surface or lifting the vacuum cleaner off the surface will cause at least one of the switches to be out of contact with the surface, thereby opening the electrical circuit and disconnecting the UV lamp fro the main power source.

6. A vacuum cleaner for cleaning a surface and sterilizing the surface at the same time as it is being cleaned, the vacuum cleaner comprising:
(a) a housing,
(b) a motor inside the housing,
(c) a main power switch for electrically connecting the motor to an external source of power,
(d) the housing having a main section and a brush section,
(e) a brush assembly in the brush section and coupled to the housing,
(f) a suction fan on the motor,
(g) a UV lamp inside the main section of the housing for emitting UV radiation,
(h) an electrical circuit for electrically connecting the main poser switch to the UV lamp, the electrical circuit comprising a track of conductive material having a plurality of gaps and a plurality of interlock switches for bridging the gaps, one interlock switch being associated with each gap,
(i) each interlock switch being open when it is out of contact with the surface and being closed when it is in contact with the surface, each interlock switch having a cap, a contact of conductive material fixed to the cap and a spring, the spring urging the cap down to a non-depressed position, the cap being disposed below the track and being movable between an up position wherein the cap is depressed and the contact bridges the gap and a down position wherein the cap is non-depressed and where the contact is spaced fro the gap,
(j) wherein, when the cap in each one of the interlock switches is in its depressed position all the gaps in the track will be closed thereby enabling current to flow in the track fro the main power switch to the UV lamp and, wherein the gaps are disposed about the housing such that tilting the vacuum cleaner in any direction relative to the surface or lifting the vacuum cleaner off the surface will cause at least one of the caps to be moved to its non-depressed position along with its associated contact, thereby opening the electrical circuit and disconnecting the UV lamp from the main power source.

7. An electric vacuum cleaner comprising:
(a) a housing,
(b) a plurality of wheels on the housing for supporting the vacuum cleaner on a surface being cleaned,
(c) a main power switch for electrically connecting the electric vacuum cleaner to an external source of power,
(d) a motor inside the vacuum cleaner housing and electrically connected to the main power switch,
(e) an impeller on the motor for creating suction inside the vacuum cleaner housing,
(f) a brush coupled to the motor,
(g) a UV light assembly mounted inside the main housing and being urged downward toward the surface, the UV light housing assembly including a UV lamp for sterilizing the surface at the same time as it is being cleaned,
(h) a UV gasket assembly fixedly mounted underneath the UV light assembly,
(i) the UV gasket assembly including a grate,
(j) the grate having an opening through which light from the UV lamp can
(k) an electrical circuit on the grate for electrically connecting the main power switch to the UV lamp, the electrical circuit comprising a track of conductive material having a plurality of gaps and a plurality of interlock switches for closing the gaps, one interlock switch being associated with each gap,
(l) each interlock switch having a cap, a contact fixed to the cap and a spring, the cap being movable up and down between a depressed position wherein the contact bridges the gap and a non-depressed position where the contact is spaced fro the gap, the spring urging the cap downward toward its non-depressed; position,
(m) wherein, when the cap in each one of the interlock switches is in its depressed position all the gaps in the track will be closed thereby enabling current to flow in the track from the main power switch to the UV lamp and, wherein, tilting the vacuum cleaner in any direction relative to the surface being cleaned or lifting the vacuum cleaner off the surface being cleaned will cause at least one of the caps to be moved to its non-depressed position along with its associated thereby opening the electrical circuit contact thereby disconnecting the UV lamp from the main power source.
